(19) United States
(12) Patent Application Publication
(10) Pub. No.: US 2021/0275031 A1
(43) Pub. Date: Sep. 9, 2021

(54) MINIATURE FLEXIBLE THERMOMETER FOR CONTINUOUS MEASUREMENT OF HUMAN BODY TEMPERATURE AND METHOD FOR MEASURING HUMAN BODY TEMPERATURE WITH THIS THERMOMETER

(71) Applicant: Pears Health Americas LLC, Charlottesville, VA (US)

(72) Inventors: Petr Skoda, nad Labem (CZ); Jaromir Krotky, Kobylisy (CZ)

(73) Assignee: Pears Health Americas LLC, Charlottesville, VA (US)

(21) Appl. No.: 17/259,478

(22) PCT Filed: Oct. 23, 2019
(86) PCT No.: PCT/CZ2019/050049

§ 371 Date: Jan. 11, 2021

(30) Foreign Application Priority Data
Oct. 24, 2018 (CZ) PV 2018-573

(51) Int. Cl. A61B 5/01; G01K 13/20; A61B 5/00; G01K 1/08; G01K 1/024

(52) U.S. Cl. CPC A61B 5/01; G01K 13/20; A61B 5/0008; G01K 1/08; A61B 2562/0271; A61B 2503/40; A61B 2562/0219; A61B 2562/166; G01K 1/024

(57) ABSTRACT

The present invention relates to a miniature flexible thermometer for continuous measurement of the human body temperature with wireless transmission, which by its design is optimized for rapid temperature response, comfortable body wear and good signal transmission. The miniature flexible thermometer according to the invention comprises a casing, inside the housing a flexible printed circuit board on which a temperature sensor, a microcontroller, a transmitter and an antenna are placed, the casing being flexible and flat in shape comprising a body and an elongated neck extending therefrom, wherein the temperature sensor is located inside the casing at the distant end of the neck. The antenna is adapted to communicate with an external electronic device and is located at the end of the body far from the temperature sensor. A small bonding surface is defined on the underside of the thermometer casing for receiving a double-sided adhesive patch. The small adhesive surface improves user comfort and at the same time it contributes to increased measurement accuracy. The invention further provides a method for determining the correct temperature in continuous stream of temperature measurements, that is, the body temperature measured by the thermometer of the invention under the correct conditions.

### CROSS-REFERENCE TO RELATED APPLICATIONS

The present application is a National Stage application under 35 U.S.C. § 371 of International Application No. PCT/CZ2019/050049 (WO 2020/083410), filed Oct. 23, 2019 which application relies on the disclosure of and claims priority to and the benefit of the filing date of Czech Application No. CZ 2018-573 (CZ2018573A3), filed Oct. 24, 2018, the disclosures of which are hereby incorporated by reference in their entireties.

### BACKGROUND OF THE INVENTION

### Field of the Invention

The present invention relates to a miniature flexible thermometer for continuous measurement of human body temperature with wireless transmission, which is optimized by its design for rapid temperature response, comfortable body wear, and good signal.

### Description of the Related Art

Human body temperature is an important biological indicator of human health and its measurement is used to quickly identify whether the body is in a normal state or is undergoing some pathological process (e.g. inflammation, infectious disease). In the case of patients with, for example, immunodeficiency, or children, it is often necessary to monitor the temperature continuously and, if necessary, to inform the nursing staff or parents immediately of any adverse temperature development. For this reason, a number of flat electronic thermometers have been recently developed which can be adhered to the body (as a patch) and contain, in addition to a temperature sensor and the necessary microelectronics, means for wireless communication with a remote electronic device (computer, tablet, smartphone), most often based on Bluetooth technology.

However, marketed thermometers of this type have a number of unresolved problems and drawbacks. One of them is the inaccurate temperature determination (whether due to improper placement of the thermometer on the human body, due to the placement of the temperature sensor in the thermometer body or due to mathematical approximation of temperature improper for the situation). Another frequent drawback is the lack of comfort for the user—either the thermometer is not flexible or it is flexible but its size is too large, or even in the case of a small size of the thermometer itself, the adhesive pad that attaches the thermometer to the body is too large. Large size in combination with an airtight pad is often a cause of excessive skin irritation during prolonged application (a day to several days) of the thermometer. The transmission of the Bluetooth signal, when placing the thermometer in axilla, is also often problematic.

For example, patent application US 2018/0028069 discloses a flexible electronic thermometer in the form of patch with wireless signal transmission, and solves some of the above problems by a breathable pad and further by placing a temperature sensor in a metal cup whose bottom is in contact with the skin, which in combination with the internal design should minimize heat losses in heat transfer between the skin and the sensor, and thus provide accurate temperature data.

Another example is patent application US 2018/0172520, which discloses a flexible electronic thermometer with wireless signal transmission comprising a base with an adhesive layer and a temperature sensor, which is covered by a cover layer with an opening into which a removable module with the appropriate electronics is placed, the module being connected via the connecting terminal to the temperature sensor.

Patent application WO 2016/108888 A1 presents another continuous thermometer of rectangular shape made of flexible rubber that adheres to human body by adhesive surface copying the rectangular outline of the device with intent to have the area of the temperature sensor firmly adhered to an axilla area. The size of device is 99×49×2.5 mm, which makes wearing the device uncomfortable. The temperature sensor is located near one of the corners but it is in close proximity to batteries having high thermal capacity. As a result, the measured temperature is affected by the temperature of a patient's surroundings, which cools down parts of batteries further away from the axilla and makes the temperature sensor cooler than the axilla area. Moreover, it takes more time to heat the temperature sensor up to a temperature of a human body in the axilla area.

Finally, patent application CN 106 137 144 A discloses another wireless flexible continuous thermometer of rectangular shape with an adhesive area covering the entire bottom side of the device again with an intent to adhere an area of the device, enclosing the temperature sensor, to an axilla area of a patient's body. Being a size of around 25×70 mm, the device adhered to the human body in the axilla area is uncomfortable and the temperature sensor is once again close to the center of one of the shorter edges, this device has similar problems with cooling of the temperature sensor by surroundings of the patient's body mentioned in the previous paragraph(s).

The present invention represents a different and improved approach to overcome the above-mentioned drawbacks; among other things, it improves measurement accuracy and signal transmission due to its design, and it improves user comfort due to its small overall size. Moreover, the present invention, contrary to what is seen in existing devices, addresses problems of patient's discomfort by using a more limited adhesive area in combination with a temperature sensor thermally separated from the rest of the thermometer body. The position of the adhesive area allows for a slight rotation of the device in a way that the temperature sensor in most positions of the human body, especially in most positions of the arm against body, resides in the axilla area; thus, the measured temperature more precisely reflects the so-called core body temperature, which is a temperature best reflecting the state of a human body.

### SUMMARY OF THE INVENTION

The subject of the present invention is particularly a flexible thermometer for continuous measurement of the human body temperature with wireless transmission optimized for rapid temperature response, comfortable wear on the body and a good signal, wherein the thermometer is attached to the human body by an extremely small adhesive surface.

The thermometer according to the invention comprises a casing in which a printed circuit board with electronic components is placed. The casing has in general flat shape where a thin neck protrudes from a relatively wide body of the thermometer, the temperature sensor being positioned inside the casing at the distant (i.e., distant from the thermometer body) end of the neck in order to minimalize the influence of the temperature of the thermometer body itself. The thermometer body may be for example approximately circular, rectangular, or oval in shape. In a preferred embodiment, the entire casing which includes the neck and the body has flat “bottle” shape.

The casing may be comprised of upper and lower parts which are joined by gluing. The entire casing of the thermometer, including the neck, is made of a flexible material (such as medical silicone rubber) so that the device does not push or obstruct in various positions when the user moves or sleeps. In another embodiment, the casing may be formed, for example, as a single corpus, wherein the internal components are directly encapsulated by the mass of the casing. The sensor for temperature measurement (hereinafter also referred to as the temperature sensor) is in direct contact with the lower part of the neck (i.e. the skin-facing part), which is as thin as possible at this position. This ensures rapid response of the temperature sensor. The position of the temperature sensor in the flexible casing is secured by a protrusion in the upper part of the casing that faces the inside of the casing and is located at the distant end of the neck, i.e. in a position corresponding to the location of the temperature sensor. The protrusion pushes the temperature sensor to the bottom of the casing, which includes at the distal end of the neck a cup-like ending with a very thin bottom for ensuring rapid heat transfer. In addition, the protrusion defines an air heat-insulating cavity in the vicinity of the temperature sensor so that it is affected by the temperature of the upper neck portion as little as possible. In addition, the temperature sensor is thermally insulated from other parts of the thermometer by one or preferably by a plurality of air cavities over the entire length of the neck. These air cavities ensure maximum thermal insulation of the temperature sensor and at the same time they provide sufficient protection of the flexible printed circuit board from bending damage. The heat insulating air cavities may optionally be replaced or filled with other insulating material, in particular in a single corpus design.

All electronic components (including temperature sensor) are located on a flexible printed circuit board. The basic components are, in addition to the temperature sensor, a microcontroller (MCU) and a transmitter with an antenna. The transmitter is a digital transmitter, i.e., a data transmission transmitter such as WiFi, Z-Wave, XBee, ZigBee, LoRa, SigFox and others, preferably a Bluetooth Low Energy transmitter. In a preferred embodiment, the transmitter may be integrated in the MCU. A precision digital or analog temperature sensor is used as a temperature sensor. The data measured by the temperature sensor are fed into the MCU, which is programmed to receive data from the temperature sensor and to deliver the data to the transmitter. The antenna is located at the opposite end of the thermometer body distant from the neck, as far as possible from the temperature sensor. This arrangement achieves maximum transmission range because the antenna protrudes from the grip between the chest and arm during temperature measurement in many positions of the human body. The antenna is tuned to achieve the best properties when the thermometer is placed on the body.

A battery holder is also placed on the printed circuit board. In one embodiment, this holder can, among other things, reinforce the thermometer body so that the thermometer's flexibility (since it consists of a flexible casing and a flexible printed circuit) is not excessive and thus does not damage the thermometer hardware.

In addition, in one embodiment the thermometer structure can be reinforced by a stiffening board, i.e. stiffener. Stiffener is a slice of a rather firm plastic (PET film) carved in the shape corresponding to the part of the thermometer casing to be reinforced. In this part, the thermometer can only be bent into a slight arc. The stiffener is located between the lower part of the thermometer casing and the printed circuit board.

On the underside of the casing or on the underside of the lower casing part, only in the area of the thermometer body, is defined an adhesive bonding surface which is extremely small (as compared to prior art thermometers) and it is located approximately at the center of the length of the thermometer body. The adhesive bonding surface area is less than 500 mm², preferably less than 400 mm². In a preferred exemplary embodiment, it is approximately 317 mm². A strong double sided adhesive patch is placed on this surface. The shape of the thermometer together with a small adhesive surface allows the thermometer to rotate slightly when the axilla position changes in different arm and shoulder positions relative to the body, so that the thermometer adhered to very elastic underarm skin causes minimal “pulling” and discomfort on the skin. In most of the arm positions with respect to the body the thermometer rotation assures pointing the thermometer neck just into the axilla and thus the position of the neck tip with a temperature sensor to measure the temperature directly in the axilla. The thermometer is placed on the chest slightly obliquely (the end of the thermometer with the antenna points down from the horizontal line of a standing person in an angle approx. 30°), thanks to which the temperature sensor is in the right place in the axilla and at the same time it does not push. The thermometer may also include other electronic sensors, for example an accelerometer. The accelerometer is primarily used to determine patient activity and body position, for example, to indicate a fall, or to indicate a change in posture or restless sleep in a recumbent patient.

In another embodiment, the thermometer may comprise an additional temperature sensor. This secondary temperature sensor can be used, for example, to detect whether the thermometer is positioned in the correct position on the body and whether the body temperature is measured correctly.

The thermometer communicates via an antenna with an external electronic device, such as a computer, tablet, or smartphone.

The aforementioned electronic device, preferably a smartphone, comprises standard hardware and software components known to those skilled in the art, and allows reception of a wireless (preferably Bluetooth Low Energy) signal from the thermometer according to the invention. The temperature data transmitted from the thermometer may be stored in the MCU memory and/or preferably in the memory of the electronic device, and by means of a computer program (application) implemented in the electronic device, the data may be processed, evaluated and presented to the user, medical staff or caregiving person. This software, in turn, can be used to control temperature monitoring with a thermometer according to the invention.

The present invention further relates to a method for determining the correct temperature, that is, the body temperature measured by the above-described thermometer according to the invention under the correct conditions. The methods used so far for non-continuous thermometers were based on waiting for thermal equilibrium and heating curve prediction. Neither of these methods can be used in continuous measurement, as both methods depend on monitoring the heating of a temperature sensor of a given thermal capacity and with a given thermal conductivity (especially for prediction) and they need sufficient thermal gradient at the beginning of the measurement for proper function. However, in a continuous measurement, the thermometer heats up from a starting temperature with sufficient thermal gradient only at the beginning of the measurement. Consequently, not all values measured during continuous measurement are correct. For example, the correct measurement in the axilla is dependent on the covering of both the axilla and the side of the chest by the upper arm, since only then does the axilla reach the temperatures that correlate with the core body temperature. At the same time, the right conditions must last long enough for the tissues around the axilla to warm up. Therefore, to avoid the many risks associated with incorrectly measured temperatures during continuous measurement, it is necessary to introduce new methods of measuring and evaluating the measured data, which will prevent misinterpretation of the data by the user or inform the user that the measurement is not correct and the measured temperature is not in appropriate relation to core body temperature.

Preferably, the aforementioned method according to the invention is a computer implemented method, which may, in the form of a computing module, be part of a software implemented in the MCU or preferably part of the program (application) implemented in a communicating electronic device (e.g. smartphone).

The method of determining the correct temperature according to the invention evaluates the data received from the thermometer and decides when the measurement was “correct,” i.e. when the user used the thermometer in proper way during the measurement. Measurements where the temperature rises in a defined manner or maintains a stable value or decreases naturally (without a rapid drop) are considered to be correct.

The temperature is measured at a frequency of 1 reading/measurement in 1 to 120 seconds, preferably 10 to 40 seconds, most preferably 1 reading/measurement in 15 seconds.

The method of determining the correct temperature c comprises the following steps.

For each measured temperature t in the continuous measuring interval, evaluate from the oldest to the most recent temperatures:

1. if, since the start of the measurement, the time T(l) of the last temperature drop by more than A° C. in y1 seconds has occurred before the time T(c) of the last correct temperature c (i.e., no rapid temperature drop occurred between last correct temperature c and current temperature t), or one of these two times is unknown, then:
a) if the evaluated temperature t at time T(t) is greater than or equal to the last correct temperature c at time T(c), then the evaluated temperature t is also the correct temperature and hence it is the new correct temperature c;
b) if the standard deviation of all temperatures in the last y2 seconds before time T(t) is less than or equal to z (i.e. the temperature is stable), then the evaluated temperature t at time T(t) is also the correct temperature and hence it is the new correct temperature c;
c) if the correct temperature c does not exist in continuous data before time T(t), then the evaluated temperature t at time T(t) is also the correct temperature and hence it is the new correct temperature c;

2. if, from the start of the measurement, the time T(l) of the last temperature decrease by A° C. y1 seconds has occurred simultaneously or later than the time T(c) of the last correct temperature c, then:
a) if the evaluated temperature t is greater than the last correct temperature c, then the evaluated temperature t at time T(t) is also the correct temperature and hence it is the new correct temperature c;
b) if all recorded temperatures in the last y2 seconds before time T(t) do not decrease in time and simultaneously the temperature s, where for time T(s) of temperature s holds T(s)=T(t)−y2, is by more than A° C. higher than the temperature r in time T(r), here T(r)=T(s)−y1 (i.e., the thermometer was covered for a long time after a rapid rise and the temperature continued to rise), then the evaluated temperature t in time T(t) is also the correct temperature and hence it is the new correct temperature c;
c) if the temperature t at time T(t) is more than A° C. higher than the temperature r at time T(r)=T(t)−y1 and simultaneously the time T(c) of the last correct temperature c occurred more than y3 hours before time T(t), then the evaluated temperature t at time T(t) is also the correct temperature and hence it is the new correct temperature c;

3. in all other cases, the measured temperature t is not considered to be the correct temperature c in terms of the correct use of the thermometer, i.e. the correct placement and heating of the thermometer without external influences such as the user's raised hand and the like.

The values of the temperature difference A° C. may be in the range of 0.05 to 0.5° C., preferably 0.15° C. The values of the time interval y1 seconds can be in the range of 30 to 160 seconds, preferably 48 seconds. The values of the time interval y2 seconds can range from 60 to 600 seconds, preferably 180 seconds. The values of the time interval y3 may be in the range 0.5 to 2 hours, preferably 1 hour. The standard deviation z may be in the range of 0.03 to 0.2° C., preferably 0.0625° C.

In an embodiment of the thermometer with two temperature sensors, where the main sensor is located at the distant end of the neck as described above, and the second, secondary sensor is located approximately halfway through the neck, and is optionally partially thermally separated from the underside of the neck, the difference in the rate of temperature rise or fall between the main and secondary sensors may be used to determine the magnitude of the temperature influence detected by the main sensor caused by the temperature of the rest of the thermometer body, and thus to more accurately determine whether the correct temperature (in the meaning as described above) was measured.

Consequently, the present invention relates to the miniature flexible thermometer for continuous measurement of the human body temperature as described above and as defined in the appended claims.

The present invention also relates to the method of measuring the body temperature by the above-described thermometer of the invention and determining the correct temperature as described above and as defined in the appended claims.

The present invention further relates to the computer-implemented method of determining the correct value of a human body temperature in a continuous measurement as described above and defined in the appended claims.

The above described method of determining the correct value of a human body temperature in a continuous measurement improves upon the state of the art by determining the correct measurements not based on maximums in time but based on, e.g., physical properties of a given measuring device and based on behavior of patients during longtime continuous measurements. This presented method overcomes problems of prior-attempted continuous temperature measurements that cannot be solved using conventional methods known from one-time measurement thermometers for human temperature measurements like “peek and hold” or temperature equilibrium prediction based on known thermal capacity of the system.

Further, the present invention, in contrast to the state of the art, uses-instead of an “as big as possible” bonding area-a relatively smaller bonding area where the portion of device enclosing the temperature sensor is, in aspects, intentionally not bonded to a human body. This way, the comfort of a patient is improved and, at the same time, the device can, thanks to placement of the bonding area near the center of the device, rotate during a patient's movements, ensuring that the temperature sensor is (most of the time) as close to the axilla area as possible, regardless of movement of the skin or limb in the axilla area. Moreover, the thermometer described herein improves upon the state of the art by, among other things, thermal separation of the temperature sensor from the rest of the device and from an upper side of the sensor's enclosing, in aspects. This minimizes the cooling of the temperature sensor by a patient's surroundings and allows for temperature measurement correlated with the core body temperature.

The present invention will now be described and explained in detail by way of examples of a preferred embodiment with reference to the accompanying drawings.

### BRIEF DESCRIPTION OF THE DRAWINGS

The accompanying drawings illustrate certain aspects of some of the embodiments of the present invention, and should not be used to limit or define the invention. Together with the written description the drawings serve to explain certain principles of the invention.

FIG. 1: Schematic representation of the thermometer viewed from the top (left figure) and viewed from the bottom (right figure).

FIG. 2: A schematic view of the arrangement of electronic components (temperature sensors, microcontroller, accelerometer, antenna and battery holder) on the printed circuit board.

FIG. 3: Spatial expansion diagram of an embodiment of the thermometer with stiffener, where the location of the stiffener between the flexible printed circuit board and the lower part of the housing is demonstrated.

FIG. 4: A view of the inside of the top part of the casing, demonstrating a protrusion for pressing the temperature sensor against the bottom of the casing and insulating air cavities.

FIG. 5: Upper panel shows a schematic representation of the correct positioning of the thermometer in axilla, lower panel is a photograph of the real situation.

FIG. 6: Graph of the time-course of temperature measurement with indication of correctly measured temperature. The temperature was measured with a frequency of 15 s for approximately 10 hours. Values marked o were detected as correct, x-marked values were detected as incorrect. Some data points are omitted in the graph for better readability.

### DETAILED DESCRIPTION OF VARIOUS EMBODIMENTS OF THE INVENTION

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Embodiments comprising various features may also consist of or consist essentially of those various features. Other embodiments of the invention will be apparent to those skilled in the art from consideration of the specification and practice of the invention. The description of the invention provided is merely exemplary in nature and, thus, variations that do not depart from the essence of the invention are intended to be within the scope of the invention. All references cited in this specification are hereby incorporated by reference in their entireties.

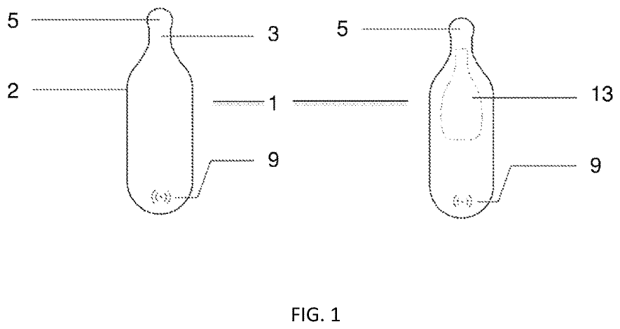

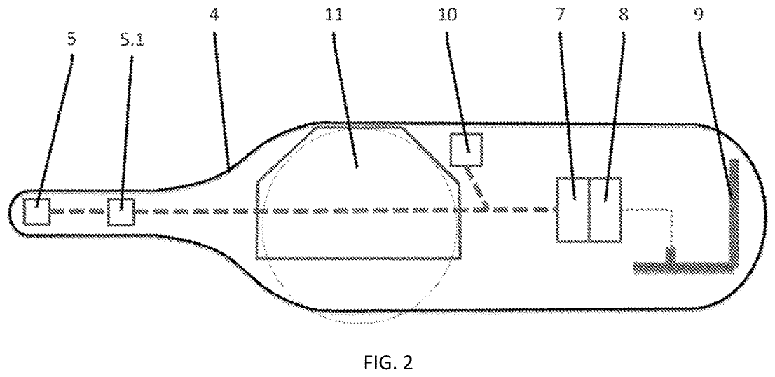

According to an embodiment of the present invention, the thermometer is shown schematically in FIG. 1 and then in more detail in FIGS. 2 to 4, wherein the thermometer comprises a casing 1 in which a printed circuit board 4 with electronic components is located. In aspects, the casing 1 has a flat bottle shape where a neck 3 protrudes from a wider oval body 2 of the thermometer, wherein the temperature sensor 5 may be located inside the casing 1 at the end of the neck 3 distant from the thermometer body 2. In aspects, the casing 1 size may be 76.9 mm×21.7 mm×5.7 mm (length, including neck×width×thickness), although the sizing may vary in embodiments.

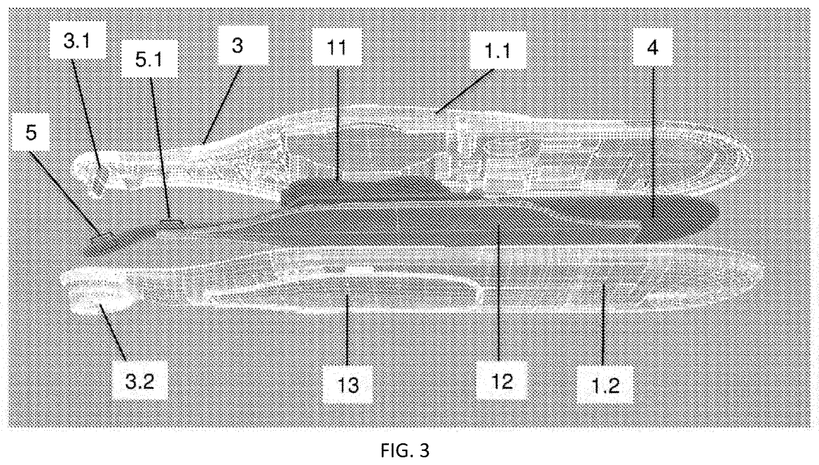

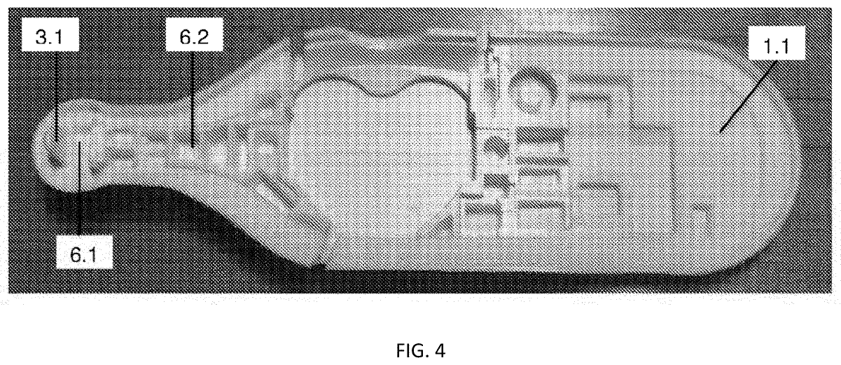

In embodiments, the casing 1 comprises an upper part 1.1 and a lower part 1.2. The thermometer casing 1, including the neck 3, is, in aspects, made of a flexible material, such as medical silicone. The upper portion 1.1 and the lower portion 1.2 are pieced together by, in aspects, gluing with a silicone adhesive. In aspects, the temperature sensor 5 (see, e.g., FIG. 3) is, through solder and metal plating on a printable circuit board, in direct contact with the lower part 3.2 of the distant end of the neck 3, which is shaped into a cup-like ending 3.2 in this part and has a thin or lowest possible thickness, such as around 0.65 mm, where it adjoins to the temperature sensor 5. The position of the temperature sensor 5 is secured by a protrusion 3.1 in the upper casing part at the distant end of the neck 3, where the protrusion 3.1 points to the interior of the casing 1 and is positioned at a position corresponding to the location of the temperature sensor 5. The protrusion 3.1 presses the temperature sensor 5 at the distant end of the neck 3 to the bottom of the cup-shaped ending 3.2 of the neck 3 and at the same time it defines a thermal insulation cavity 6.1 (see FIG. 4) above the temperature sensor 5 to prevent thermal influence of the temperature sensor 5 from the top of the housing. In addition, the temperature sensor 5 is thermally insulated from the other parts of the thermometer by a plurality of air cavities 6.2 between the upper part and the lower part 1.2 of the casing 1 over the entire length of the neck 3, in examples.

Electronic components (including temperature sensor 5) can be placed on a flexible printed circuit board 4 (e.g., a polyimide film with a copper layer, varnish and a surface finish of the wiring). The basic components are, in addition to said temperature sensor 5, a microcontroller (MCU) 7, a transmitter 8 and an antenna 9.

In embodiments, a microcontroller 7 with an integrated transmitter 8 is used. In aspects, antenna 9 is a type of planar inverted F (PIFA) antenna of a small size adapted for a given substrate and location on the human body.

In embodiments, an accurate digital temperature sensor is used as a temperature sensor 5, which converts the measured signal corresponding to the temperature into digital form. In embodiments, the signal measured by the temperature sensor 5 is fed into the MCU 7, where it is processed and optionally stored in a memory, and then inside the MCU 7, the signal is fed into the transmitting part (transmitter 8). The antenna 9 is located at the end of the thermometer body 2 distant from the neck 3, as far as possible from the temperature sensor 5, in aspects.

In this particular embodiment, a second temperature sensor 5.1 is located in or around a middle section of the neck 3.

Further, in this particular embodiment, an accelerometer 10 is added to the thermometer electronics. In aspects, a battery holder 11 is also placed on the printed circuit board 4. In aspects, a battery CR1620 (3.0 V) is used.

In addition, in this embodiment, the thermometer structure is further reinforced by a stiffener 12 cut from PET film or similar material in the form of a corresponding portion of the thermometer casing 1 to be reinforced. The stiffener 12 may be located between the lower part 1.2 of the thermometer casing and the printed circuit board 4.

Further, standard electronic components (pushbutton, LED, capacitors, resistors, antenna circuit, etc.) may be used on the printed circuit board 4 which are known to those skilled in the art and all components (including temperature sensor 5, MCU 7 with transmitter 8 and antenna 9) are connected in a substantially standard manner known to those skilled in the art.

At the underside of the lower part 1.2 of the casing 1, an adhesive bonding surface 13 may be defined by a contoured edge. The adhesive bonding surface 13 may be approximately 317 mm², in aspects, and may be located approximately at the center of the length of the thermometer casing 1. A double-sided adhesive patch may be attached to this surface 13.

In aspects, the thermometer communicates (bi-directionally) via the antenna 9 with an external electronic device, computer, tablet or smartphone, internet, server, or cloud where software (application) for receiving, recording and processing the measured data is installed.

### EXAMPLE 2

Method of Determining the Correct Temperature

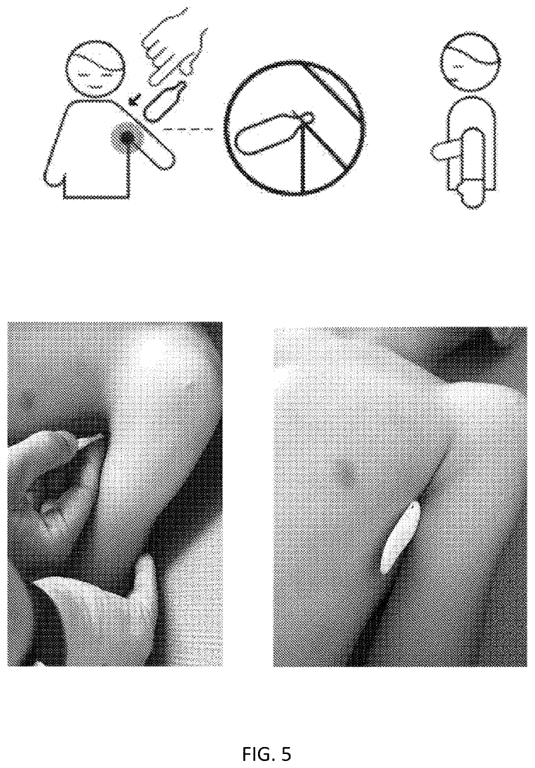

The thermometer is placed on the chest slightly obliquely (the end of the thermometer casing 1 with the antenna 9 points approximately 30° down from the horizontal line of a standing person) so that the distant end of the neck 3 with the temperature sensor 5 lies in the axilla (see FIG. 5).

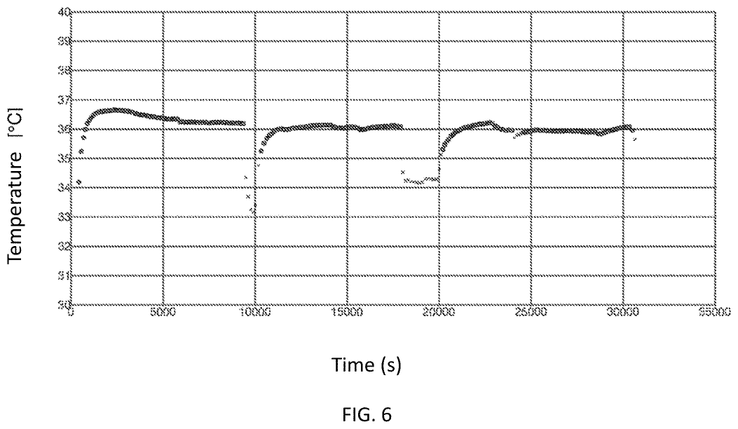

The term correct temperature c means the temperature measured by the thermometer under the right conditions when the user used the thermometer in the correct way. The temperature was measured continuously (see FIG. 6) over time, with a frequency of 15 s, the temperature measurements t were considered to be correct when the temperature t rose in a defined manner or maintained a stable value or decreased naturally (not a sharp drop occurred).

An exemplary method of determining the correct temperature c measured by the thermometer of Example 1 involved the following steps.

For each measured temperature t at time T(t) within the continuous measuring interval evaluate:

1. if the time T(l) of the last temperature drop by more than 0.15° C. in 48 seconds occurred before the time T(c) of the last correct temperature c, or one of these times is unknown, then:
a) if the evaluated temperature t at time T(t) is greater than or equal to the last correct temperature c at time T(c), then the evaluated temperature t at time T(t) is the new correct temperature c;
b) if the standard deviation of the temperatures over the last 180 seconds before time T(t) is less than or equal to 0.0625° C., then the evaluated temperature t at time T(t) is the new correct temperature c;
c) if the correct temperature c does not exist in the continuous data, then the evaluated temperature t at time T(t) is the new correct temperature c;

2. if the time T(l) of the last drop of temperature by at least 0.15° C. in 48 seconds occurred simultaneously or later than time T(c) of the last correct temperature c, then:
a) if the temperature t is greater than the last correct temperature c, then the evaluated temperature t at time T(t) is the new correct temperature c;
b) if all temperatures in the last 180 seconds before time T(t) have not decreased and simultaneously the temperature increased by more than 0.15° C. within 48 second interval before 180 seconds before time T(t), then the evaluated temperature t at time T(t) is the new correct temperature c;
c) if the temperature t increased by more than 0.15° C. in the last 48 seconds and simultaneously the time T(c) of the last correct temperature c is older than 1 hour before time T(t), then the evaluated temperature/at time T(t) is the new correct temperature c;

3. in all other cases the measured value t is not considered to be the correct temperature c.

One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. A flexible thermometer for continuous measurement of human or animal body temperature comprising:
a temperature sensor;
a casing comprising a body portion and a flexible neck portion, wherein the flexible neck portion contains the temperature sensor and comprises a first air cavity adjacent to the temperature sensor, and wherein the body portion comprises at least one second air cavity;
one or more circuit boards;
a microcontroller a transmitter; and
an antenna;
wherein the one or more circuit boards are in operable communication with the temperature sensor, the microcontroller, the transmitter, the antenna, or combinations thereof, wherein the microcontroller or the transmitter are capable of receiving a signal from the temperature sensor, and wherein the antenna is capable of communicating temperature or other information to an external electronic device; and
wherein the flexible thermometer, the flexible neck portion, or both are capable of rotating, moving, or bending when the flexible thermometer is attached to a human or animal.

2. (canceled)

3. The flexible thermometer according to claim 1, wherein the casing comprises an adhesive surface, a double-sided adhesive, a bonding surface, or a cohesive surface.

4. (canceled)

5. The flexible thermometer of claim 1, further comprising an accelerometer and/or a gyroscope.

6. The flexible thermometer according to claim 1, further comprising a stiffening member located between the one or more circuit boards and an internal surface of the casing.

7. The flexible thermometer according to claim 1, further comprising at least one ether second temperature sensor.

8. A method for measuring of a human body temperature, the method comprising the steps:
providing a thermometer capable of measuring temperature;
for each measured temperature t in a continuous measuring interval, evaluated from an oldest or last to a latest temperature measurement,
1. if an evaluated temperature t at time T(t) is greater than or equal to a last correct temperature c at time T(c), then the evaluated temperature t is a new correct temperature c;
2. if, since a start of a measurement, a time T(l) of the last temperature drop by more than x° C. in y1 seconds has occurred before the time T(c) of the last correct temperature c, or T(l) is unknown, then: if a standard deviation of all temperatures in y2 seconds before time T(t) is less than or equal to z, then the evaluated temperature t at time T(t) is the new correct temperature c.

9. (canceled)

10. (canceled)

11. The flexible thermometer according to claim 1, wherein the flexible neck portion is at least 10 millimetres in length.

12. The flexible thermometer according to claim 1, wherein the antenna is at an end of the casing opposite from where the body portion connects to the flexible neck portion.

13. The flexible thermometer according to claim 1, wherein the circuit board comprises a flexible material.

14. The flexible thermometer according to claim 13, wherein the temperature sensor is located on the circuit board.

15. The flexible thermometer according to claim 1, wherein the temperature sensor is located in an area of the flexible neck portion distant from where the flexible neck portion connects to the body portion.

16. The flexible thermometer according to claim 3, wherein the adhesive surface, the double-sided adhesive, the bonding surface, or the cohesive surface is less than 500 mm².

17. The flexible thermometer according to claim 3, wherein the adhesive surface, the double-sided adhesive, the bonding surface, or the cohesive surface covers less than 40% of a surface of the casing of the flexible thermometer.

18. The flexible thermometer according to claim 3, wherein the adhesive surface, the double-sided adhesive, the bonding surface, or the cohesive surface covers less than 35% of a surface of the body portion of the casing.

19. The flexible thermometer according to claim 3, wherein the adhesive surface, the double-sided adhesive, the bonding surface, or the cohesive surface is located on a side of the casing to be attached to a user, wherein the adhesive surface, the double-sided adhesive, the bonding surface, or the cohesive surface is capable of allowing the flexible thermometer to bend, move, or rotate when attached to the user.

20. The flexible thermometer according to claim 3, wherein the adhesive surface, the double-sided adhesive, the bonding surface, or the cohesive surface is capable of allowing the flexible thermometer to bend, move, or rotate when attached to a user, and wherein the adhesive surface, the double-sided adhesive, the bonding surface, or the cohesive surface is capable of allowing the flexible thermometer to bend, move, or rotate when the user moves, when the axilla position of the user changes, or when the user changes arm or shoulder position(s).

21. The flexible thermometer according to claim 1, further comprising a double-sided adhesive capable of being attached on one side to the flexible thermometer.

22. The flexible thermometer according to claim 1, further comprising a double-sided adhesive, wherein the double-sided adhesive is attached to a side of the flexible thermometer to be adhered to a user, wherein the double-sided adhesive is capable of allowing the flexible thermometer to bend, move, or rotate when attached to the user.

23. The flexible thermometer according to claim 1, further comprising a double-sided adhesive, wherein the double-sided adhesive is capable of allowing the flexible thermometer to bend, move, or rotate when attached to a user, and wherein the double-sided adhesive is capable of allowing the flexible thermometer to bend, move, or rotate when the user moves, when the axilla position of the user changes, or when the user changes arm or shoulder position(s).

24. The flexible thermometer according to claim 1, wherein the casing is under 100 millimetres in length, under 30 millimetres in width, and under 10 millimetres in thickness.

25. The flexible thermometer according to claim 1, wherein the first air cavity is capable of thermally insulting the temperature sensor.

26. The flexible thermometer according to claim 1, wherein the temperature sensor is thermally separated or insulated from the one or more circuit boards, the microcontroller, the transmitter, the antenna, a battery, or combinations thereof.

27. The flexible thermometer according to claim 1, wherein the flexible neck portion comprises a protrusion capable of holding the temperature sensor in place within the flexible neck portion.

28. The flexible thermometer according to claim 1, wherein the first air cavity is on a side of the temperature sensor that is not in contact with or is facing away from a user or skin of a user.

29. The flexible thermometer according to claim 1, wherein the one or more circuit boards are positioned between an upper part and a lower part of the casing.

30. The flexible thermometer according to claim 1, further comprising a stiffening member located between the one or more circuit boards and a portion of the casing near to or next to the flexible neck portion.

31. The flexible thermometer according to claim 7, wherein the at least one second temperature sensor is distance separated from the temperature sensor, and a difference in a rate of temperature rise or fall between the at least one second temperature sensor and the temperature sensor, and/or a difference in a magnitude of measured temperature detected between the at least one second temperature sensor and the temperature sensor, is capable of determining whether the flexible thermometer is taking accurate temperature measurements.

32. The flexible thermometer according to claim 1, wherein a skin-facing side of the flexible neck portion comprises material having a thickness of about 0.2 millimetres to about 0.8 millimetres capable of allowing for heat transfer to the temperature sensor.

33. The flexible thermometer according to claim 1, wherein a protrusion in the flexible neck portion presses the temperature sensor against the flexible neck portion, and wherein an air-insulating cavity is formed around or near the temperature sensor.

34. The flexible thermometer according to claim 1, further comprising a processor, wherein the processor is capable of determining temperature correctness by calculating whether a continuously measured temperature is increasing or decreasing in a defined manner and/or whether a temperature maintains a stable or near stable value.

35. The flexible thermometer according to claim 1, further comprising a processor, wherein the processor is capable of determining temperature correctness by calculating whether a temperature change occurs over a set period of time that is determined by the processor to indicate that the temperature sensor is not taking correct temperature measurements.

36. The flexible thermometer according to claim 1, further comprising a processor, wherein the processor is capable of determining temperature correctness by using a starting temperature and/or known heat capacity of the flexible thermometer, the temperature sensor, the first air cavity, the casing, the flexible neck portion, the body portion, or combinations thereof

37. A method for measuring a human body temperature, comprising the steps of:
providing a thermometer;
continuously measuring temperature in regular or random measuring intervals, evaluated from an first temperature measurement to a second, more recent temperature measurement;
determining a correct temperature in continuous stream of measurements to be: 1) any temperature higher than a closest previous correct temperature, 2) any temperature where the closest previous correct temperature is closer than closest previous temperature drop by more than x° C. in a predetermined amount of time, and having a standard deviation of temperature measurements in preceding predetermined amount of time less than or equal to a predetermined limit, 3) any temperature where all preceding temperature measurements in a predefined amount of time do not decrease and at the same time the interval of not decreasing temperatures was preceded by a temperature rise by more than x° C. in a predefined amount of time, or 4) any temperature preceded by a temperature rise by more than x° C. in a predefined amount of time and for which a closest previous correct temperature is older than a predefined amount of time.

38. The method for measuring a human body temperature according to claim 8, wherein if all recorded temperatures in y2 seconds before time T(t) do not decrease in time and simultaneously a temperature s, where for time T(s) of temperature s holds T(s)=T(t)−y2, is by more than x° C. higher than a temperature r in time T(r), where T(r)=T(s)−y1, then the evaluated temperature t in time T(t) is the new correct temperature c.

39. The method for measuring a human body temperature according to claim 8, wherein if temperature t at time T(t) is more than x° C. higher than a temperature r at time T(r)=T(t)−y1 and simultaneously a time T(c) of a last correct temperature c occurred more than y3 hours before time T(t), then the evaluated temperature t at time T(t) is the new correct temperature c.

40. The method for measuring a human body temperature according to claim 39, wherein in all other cases, the temperature t is not considered to be the correct temperature c.

41. The method for measuring a human body temperature according to claim 37, wherein a temperature difference value x is in a range 0.05 to 0.5 ° C.

42. The method for measuring a human body temperature according to claim 39, wherein the value of time interval y1 is in a range of 30 to 160 seconds, the value of time interval y2 is in a range of 60 to 600 seconds, the value of time interval y3 is in a range of 0.5 to 2 hours, and the standard deviation value z is in a range of 0.03 to 0.2° C.

43. The method for measuring a human body temperature according to claim 37, wherein the standard deviation value is in a range of 0.03 to 0.2° C.

44. The method for measuring a human body temperature according to claim 37, wherein a temperature measurement is measured at a frequency of 1 reading per 1 to 120 seconds.
